(19) United States
(12) Patent Application Publication
(10) Pub. No.: US 2009/0269334 A1
(43) Pub. Date: Oct. 29, 2009

(54) BIOMARKERS FOR GRAFT REJECTION

(76) Inventors: Marc Bigaud, Sundhoffen (FR); Jeanne Kehren, Rumersheim Le Haut (FR); Friedrich Raulf, Freiburg (DE); Grazyna Wieczorek, Schonenbuch (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/501,706

(22) Filed: Jul. 13, 2009

Related U.S. Application Data
(63) Continuation of application No. 10/581,068, filed on Nov. 9, 2006.
(60) Provisional application No. 60/526,722, filed on Dec. 3, 2003.

(51) Int. Cl. A61K 39/395; C12Q 1/68; C12Q 1/00; G01N 33/53

(52) U.S. Cl. 424/130.1; 435/6; 435/4; 435/7.1

(57) ABSTRACT

Methods are disclosed for diagnosing rejection in a transplanted subject, monitoring rejection in a transplanted subject at risk thereof, preventing, inhibiting, reducing or treating rejection in a transplanted subject, or identifying agents for use in the prevention, inhibition, reduction or treatment of rejection, based on genes which are differentially expressed in transplanted subjects.

This invention relates to a method of monitoring the status of a transplanted tissue or organ In a recipient. In particular, the Invention relates to the use of gene expression analysis to indicate allograft rejection, more particularly acute allograft rejection (AR) or chronic allograft rejection (CR). The expression of certain genes (at the RNA or protein level) may be used as a means of detecting rejection and/or describing the histological and/or pathological status of the graft.

Chronic allograft rejection is the major cause for the failure of long-term graft survival. In contrast to treatable acute rejection episodes, chronic rejection is not reversible to date by any treatment when histologically detected, is not proven to be preventable by any immunosuppressive regimen and its pathogenesis is not fully understood but involving immunological as well as non-immunological factors. Characteristic for chronic rejection in all solid organ grafts is a concentric arterial intimal thickening by vascular remodeling. Kidney allografts with chronic rejection exhibit in addition pronounced parenchymal fibrosis and glomerular sclerosis: clinically, CR is manifested by a progressive decline in renal function, accompanied by proteinuria and hypertension.

There is a need to have a reliable tool for identification, prognosis and follow-up of allograft rejection, particularly CR, preferably early prognosis of CR before any overt clinical or histological manifestation, or before loss of function of the graft, e.g. within the first year post transplantation; such an aid would be valuable e.g. for the optimization of current treatment regimens and the design of clinical trials, including with new CR inhibiting agents.

The present invention relates to the Identification of biomarkers for allograft rejection, e.g. genes which are differentially expressed in transplanted subjects, e.g. renal biopsies, before or after the onset of rejection, compared to healthy tissues (where rejection does not develop). The resulting gene expression pattern of a subset of the genes allows a highly statistically significant discrimination of the tissues undergoing CR from those undergoing AR and from healthy tissues. The complete sequences of these genes are available using the GenBank accession number or RefSeq Identifier shown in Tables 1 to 3. The sequences as shown under the corresponding GenBank accession number or RefSeq Identifier are incorporated herein by reference.

The genes identified according to the invention are useful biomakers for the identification and/or prognosis of rejection in transplanted subjects. The present invention provides a group of genes which are indicative of transplant rejection (either AR or CR, see Table 1), a group of genes which are indicative of chronic rejection (see Table 2) and a gene which is indicative of acute rejection (see Table 3). Any selection, of at least one, of these genes can be utilized as surrogate biomarker for diagnosis and/or prognosis of rejection, e.g. CR. In particularly useful embodiments, a plurality of these genes can be selected and their mRNA expression monitored simultaneously to provide expression profiles for use in various aspects.

The biomarker genes are expressed to low level in normal tissues and are expressed during rejection. In order to distinguish CR from AR, preferably two or more genes are used. The biomarkers are indicative of the status of the graft and indicate the development of the pathological changes. They can be used as more sensitive detection means before the rejection leads to a significant loss of function translated in terms of clinical detection as the increased of serum creatinine and urea (decreased glomerular filtration rate).

The genes identified in tables 1 to 3 are particularly useful as biomarkers as they are potentially detectable in a body fluid (e.g. serum, plasma or urine). Thus biopsy samples from a transplanted tissue are not necessarily required.

Accordingly, the invention provides the use of a gene as listed In Table 1, 2 or 3 as a biomarker for transplant rejection, e.g. as a biomarker for CR. Preferably one or more genes in Table 2 are used as biomarkers for CR, or indolamine deoxygenase is used as a biomarker for AR.

In a further embodiment, the levels of the gene expression products (proteins) can be monitored in various body fluids, including, but not limited to, blood plasma, serum, lymph, urine, stool and bile, or in biopsy tissues. This expression product level can be used as surrogate markers for early diagnosis of rejection and can provide indices of therapy responsiveness.

Accordingly, the invention also provides the use of an expression product of (e.g. a protein encoded by) a gene as listed in Table 1, 2 or 3 as a biomarker for (e.g. chronic) transplant rejection.

The methods of the present invention may be performed in vitro, e.g. the levels of biomarkers may be analyzed In tissues or fluids extracted or obtained from a transplanted subject.

Methods of detecting the level of expression of mRNA are well-known in the art and include, but are not limited to, reverse transcription PCR, real time quantitative PCR, Northern blotting and other hybridization methods.

A particularly useful method for detecting the level of mRNA transcripts obtained from a plurality of the disclosed genes involves either the hybridization of labeled mRNA to an ordered array of oligonucleotides or the analysis of total RNA by TaqMan low density arrays. Such a method allows the level of transcription of a plurality of these genes to be determined simultaneously to generate gene expression profiles or patterns. The gene expression profile derived from the tissues obtained from the transplanted subject at risk of developing rejection, e.g. CR or AR, can be compared with the gene expression profile derived from the sample obtained from a normal organ.

In a further embodiment, measuring expression profiles of one or a plurality of these genes or encoded proteins could provide valuable molecular tools for examining the efficacy of drugs for inhibiting, e.g. preventing or treating, rejection (e.g. changes in the expression profile from a baseline profile while the transplanted patient is exposed to therapy).

Accordingly, this invention also provides a method for screening a transplanted subject to determine the likelihood that the subject will respond to anti-rejection therapy, methods for the identification of agents that are useful In treating a transplanted subject (e.g. showing signs of CR) and methods for monitoring the efficacy of certain drug treatments for rejection, e.g. CR or AR.

The term “differentially expressed” refers to a given allograft gene expression level and is defined as an amount which is substantially greater or less than the amount of the corresponding baseline expression level. Baseline is defined here as being the level of expression in healthy tissue. Healthy tissue includes a transplanted organ without pathological findings.

In another aspect, the invention provides a (e.g. in vitro) method of monitoring transplant rejection, e.g. CR, in a test transplanted subject by detecting a differentially expressed gene in a given tissue sample. For example, the method may comprise:

a) taking as a baseline value the level of mRNA expression corresponding to or protein encoded by at least one gene, e.g. as Identified in Table 1, 2 or 3, e.g. in a specific tissue sample of a control transplanted subject who is known not to develop rejection, e.g. CR;

b) detecting a level of mRNA expression corresponding to or protein encoded by the at least one gene identified in a) in an tissue sample of the same tissue type as in a) obtained from a test transplanted subject; and

c) comparing the first value with the second value, wherein a first value lower or higher than the second value predicts that the test transplanted subject is at risk of developing rejection, e.g. CR.

According to another embodiment, the (e.g. in vitro) method may also comprise

a) detecting a level of mRNA expression corresponding to or protein encoded by at least one gene, e.g. as Identified in Table 1, 2 or 3, in an tissue sample obtained from the donor, preferably a living donor, at the day of transplantation,

b) detecting a level of mRNA expression corresponding to or protein encoded by the at least one gene identified in a) in an tissue sample obtained from a patient post-transplantation,

c) comparing the first value with the second value, wherein a first value lower or higher than the second value predicts that the transplanted subject is at risk of developing rejection.

In steps b) above, the level of mRNA or protein encoded is preferably detected within 4 to 7 months post-transplantation, more preferably around 6 months post-transplantation.

The method of diagnosing rejection, e.g. CR, according to the invention may also be applied to maintenance patients, i.e. patients who have been transplanted more than one year ago. Accordingly, tissue samples are taken and the level of mRNA expression corresponding to at least one gene is compared to the level in the reference control values to identify patients that will may developing CR.

In another aspect, the invention provides a method for monitoring, e.g. preventing or inhibiting or reducing or treating rejection, e.g. CR, in a transplanted subject at risk of developing rejection, with an inhibitor (e.g. a small molecule, an antibody or other therapeutic agent or candidate agent). Monitoring the Influence of agents (e.g. drug compounds) on the level of expression of a marker of the invention can be applied not only in basic drug screening, but also in clinical trials. For example, the effectiveness of an agent to affect marker expression can be monitored in clinical trials of transplanted subjects receiving treatment for the inhibition of rejection.

Such a method comprises:

a) obtaining a pre-administration sample from a transplanted subject prior to administration of the agent,

b) detecting the level of expression of mRNA corresponding to or protein encoded by the at least one gene in the pre-administration sample,

c) obtaining one or more post-administration samples from the transplanted patient,

d) detecting the level of expression of mRNA corresponding to or protein encoded by the at least one gene In the post-administration sample or samples,

e) comparing the level of expression of mRNA or protein encoded by the at least one gene in the pre-administration sample with the level of expression of mRNA or protein encoded by the at least one gene in the post-administration sample or samples, and

f) adjusting the agent accordingly.

For example, increased or decreased administration of the agent may be desirable to change the level of expression of the at least one gene to higher or lower levels than detected. In above method, the agent can also be administered alone or In combination with other agents in a combined therapy, preferably with immunosuppressive agents and/or agents effective In transplant rejection, e.g. AR or CR. Step f) may include the change of the treatment dose, change of regimen, change of treatment agent, or addition of one or more further agent in combination (e.g. sequentially or concomitantly) with the agent already used.

Accordingly, incorporation of gene expression profiling data from human tissue samples, will help improve the patient selection process during clinical trials aimed at both treatment and prevention of the progression of rejection, e.g. CR or AR.

In a yet other aspect, the invention further provides a method for identifying agents for use In the prevention, inhibition, reduction or treatment of transplant rejection, e.g. CR or AR, comprising monitoring the level of mRNA expression of at least one gene or protein encoded as disclosed above.

In a further aspect, the invention provides a method for preventing, inhibiting, reducing or treating transplant rejection, e.g. CR or AR in a subject in need of such treatment comprising administering to the subject a compound that modulates the synthesis, expression or activity of one or more genes or gene products, as disclosed In Table 1, 2 or 3, so that at least one symptom of rejection is ameliorated.

In a further aspect, the invention provides a compound (e.g. a small molecule, an antibody or other therapeutic agent or candidate agent) which modulates the synthesis, expression of activity of one or more genes or gene products identified above (e.g. a gene identified in Table 1, 2 or 3) for use as a medicament, e.g. for the prevention or treatment of transplant rejection in a subject.

In a further aspect, the invention provides the use of a compound (e.g. a small molecule, an antibody or other therapeutic agent or candidate agent) which modulates the synthesis, expression of activity of one or more genes or gene products identified above (e.g. a gene identified in Table 1, 2 or 3) for prevention or treatment of transplant rejection, e.g. CR in a subject.

In a further aspect, the invention provides the use of a compound (e.g. a small molecule, an antibody or other therapeutic agent or candidate agent) which modulates the synthesis, expression of activity of one or more genes or gene products identified above (e.g. a gene identified in Table 1, 2 or 3) for the preparation of a medicament for prevention or treatment of CR In a transplanted subject.

Examples of such compounds or agents are e.g. compounds or agents having immunosuppressive properties, e.g. as used in transplantation, e.g. a calcineurin inhibitor, e.g. Cyclosporin A or FK506, a mTOR inhibitor, e.g. rapamycin or a derivative thereof, e.g. rapamycin substituted in position 40 and/or 16 and/or 32, e. g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro40—O—(2-hydroxyethyl)rapamycin, 40-[3-hydroxy-2-(hydroxy-methyl)-2-methylpropanoate]-rapamycin (also called CC1779), 40-epi-(tetrazolyl)-rapamycin (also called ABT578), 40—O—(2-hydroxyethyl)-rapamycin, or a rapalog, e.g. as disclosed In WO 98/02441, WO01/14387 and WO 03/64383, e.g. AP23573, AP23464, AP23675 or AP23841, or a CCR5 antagonist, e.g. (2,4-dimethyl-1-oxy-pyridin-3-yl)-[4′-methyl-4-(phenyl-pyridin-3-yl-amino)-[1,4′]bipiperidinyl-1′-yl]-methanone. These compounds or agents may also be used in combination.

By transplanted subject is meant a subject receiving cells, tissue or organ from a donor, preferably from the same species, e.g. kidney, heart, lung, combined heart and lung, liver, pancreas (e.g. pancreatic islet cells), bowel (e.g., colon, small intestine, duodenum), neuronal tissue, limbs. The subject is preferably a human. Alternatively the method may be performed In other animals, e.g. mammals such as monkeys or rats. The method of the present invention for identifying agents for use in treating transplant rejection may advantageously be performed in monkeys, due to the high likelihood that agents identified in such a way will also be effective in humans.

Preferably more than one gene, e.g. a set of genes, are used in the methods of the invention. The methods of the invention are particularly preferred in kidney transplantation.

Gene expression profiles can be generated using e.g. the Affymetrix microarray technology. Microarrays are known in the art and consist of a surface to which probes that correspond in sequence to gene products (e.g. mRNAs, polypeptides, fragments thereof etc.) can be specifically hybridized or bound to a known position. Hybridization intensity data detected by the scanner are automatically acquired and processed by the GENECHIP^{R} software or Affymetrix microarray analysis suite software. Raw data is normalized to expression levels using a target intensity of 200.

The transcriptional state of a cell may be measured by other gene expression technologies known in the art. Several such technologies produce pools of restriction fragments of limited complexity for electrophoretic analysis, such as methods combining double restriction enzyme digestion with phasing primers (e.g. EP-A1-0 534858), or methods selecting restriction fragments with sites closest to a defined mRNA end (e.g. Prashar et al, Proc. Nat. Acad. Sci., 93, 659-663, 1996). Other methods statistically sample cDNA pools, such as by sequencing sufficient bases (e.g. 20-50 bases) in each multiple cDNAs to identify each cDNA, or by sequencing short tags (e.g. 9-10 bases) which are generated at known positions relative to a defined mRNA end (e.g. Velculescu, Science, 270, 484-487, 1995) pathway pattern.

In another embodiment of the present invention, a protein corresponding to a marker is detected. A preferred agent for detecting a protein of the invention is e.g. an antibody capable of binding to the protein, preferably an antibody with a detectable label. Antibodies can be polyclonal, or preferably, monodonal. An intact antibody or a fragment thereof (e.g. Fab or F(ab′)₂ can be used. The term “labeled” is intended to encompass direct labelling of the antibody by coupling a detectable substance to antibody, as well as indirect labeling of the antibody by reactivity with another reagent that is directly labeled. A variety of formats can be employed to determine whether a sample contains a protein that binds to a given antibody. Examples of such formats include e.g. enzyme immunoassay, radioimmunoassay, Western blot analysis and ELISA.

In a preferred embodiment, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with main memory. The external components include mass data storage. This mass storage can be one or more hard disks. Other external components include user interface device, which can be a monitor and keyboards, together with pointing device or other graphic input devices. Typically, the computer system is also linked to other local computer systems, remote computer systems or wide area communication networks, e.g. Internet. This network link allows the computer system to share data and processing tasks with other computer systems.

Several software components are loaded into memory during operation of this system. These software components collectively cause the computer system to function according to the methods of this invention. These software components are typically stored on mass storage or on removable media, e.g. floppy disks or CD-ROM. The software component represents the operating system, which is responsible for managing the computer system and Its network interconnections. Preferably the methods of this invention are programmed in mathematical software packages, which allow symbolic entry of equations and highlevel specification of processing, including algorithms to be used, and thereby freeing a user of the need to procedurally program individual equations or algorithms.

In preferred embodiments, the analytic software component actually comprises separate software components that interact with each other. Analytic software represents a database containing all data necessary for the operation of the system. Such data will generally include, but is not limited to, results of prior experiments, genome data, experimental procedures and cost, and other information, which will be apparent to those skilled in the art. Analytic software includes a data reduction and computation component comprising one or more programs which execute the analytic methods of the invention. Analytic software also includes a user interface which provides a user of the computer system with control and input of test network models and, optionally, experimental data. The user interface may comprise a drag-and-drop interface for specifying hypotheses to the system. The user interface may also comprise means for loading experimental data from the mass storage component, from removable media or from a different computer system communicating with the instant system over a network.

The invention also provides a process for preparing a database comprising at least one of the markers set forth in this invention, e.g. mRNAs. For example, the polynucleotide sequences are stored in a digital storage medium such that a data processing system for standardized representation of the genes that identify transplant rejection. The data processing system is useful to analyze gene expression between two tissue samples taken at different time point, e.g. at the transplantation day and post-transplantation. The isolated polynucleotides are sequenced. The sequences from the samples may be compared with the sequence(s) present in the database using homology search techniques. Alternative computer systems and methods for implementing the analytic methods of this invention will be apparent to one skilled in the art and are intended to be comprehended within the accompanying claims.

Identification of Diagnostic Markers of Rejection

Tissue samples from kidney-transplanted non-human primate (cynomolgus monkey) models of acute and chronic rejection are obtained. The lesions induced In these models have been examined and been found to be remarkably similar to the histological modifications observed in humans.

Acute rejection is studied In cynomolgus monkey life-supporting kidney allografts. Transplantation is associated with bilateral nephrectomy at the time of graft implantation. Animals are treated either with a suboptimal dose of cyclosporin A (Neoral®), 20 mg/kg or with (2,4-dimethyl-1-oxy-pyridin-3yl)-[4′-methyl-4-(phenyl-pyridin-3-yl-amino) -[1,4′]bipiperidinyl-1′-yl]-methanone monotherapy 20 mg/kg bid or with a combination of both compounds. Animals are sacrificed 6 to 9 days post-transplantation. Histopathological examination of grafts reveals AR in all cases.

Chronic rejection is studied in cynomolgus monkey non life-supporting kidney allografts. Transplantation is associated with unilateral nephrectomy so one native kidney is left in place. Animals are treated with an anti-rejection therapy combining anti-thymoglobulin/steroid/cyclosporin A (20 mg/kg i.v. 5 times every 2 days/10 mg/kg i.v. 5 times every 2 days/150 mg/kg/d p.o.) and are sacrificed between 44 and 147 days post-transplantation, or cyclosporin A is withdrawn on day 149 post-transplantation and animals are sacrificed between 231 and 331 days post-transplantation. Histological examination of the grafts reveals various degrees of CR.

Control kidneys are collected at the time of transplantation (uni- or bilateral nephrectomy).

Tissue Homogenization

All liquid nitrogen flash-frozen kidney cortex samples are stored in cryotubes at −80° C. Immediately after the addition of 700 μl homogenization buffer (ABI lysis buffer/PBS 1:1) the homogenization step is performed by dipping the rod of a Polytron rotor/stator homogenizer PT 3100 into the tissue containing buffer and running the homogenizer at full speed for 30 seconds. If after this time remnant tissue pieces are visible, the procedure is repeated until homogeneity is achieved. Hereafter the homogenate is stored at −80° C. until it is used in the RNA extraction step.

Homogenate Pre-Filtration and RNA Extraction

Pre-filtration of the homogenate and RNA extractions are performed by the ABI 6700 Biorobot workstation (Applied Biosystems, USA). Tissue homogenates are filled into the wells of a 96-deep-well plate, and placed in the filtrate position of the 6700 workstation. A tissue pre-filter tray is placed into the purification carriage and locked into position. The instrument door is closed, and the workstation software is launched.

The RNA extraction procedure includes a sample transfer step, a filtration step, a washing step, and an elution step. The sample transfer step, in which the pre-filtered homogenate is transferred from the 96 deep-well plate to the RNA purification tray includes a primary transfer of 550 μl solution. Before the second transfer, 150 μl homogenization buffer (Applied Biosystems lysis buffer/PBS 1:1) is added to each well in the deep-well plate, mixed three times and then 150 μl are transferred from there to the purification tray. The filtration step is carried out by applying a vacuum pressure of 80% for 180 seconds. The washing steps are performed as follows:

Step 1. washing solution 1, 400 μl, vacuum pressure 80% for 180 seconds, two times;

Step 2: washing solution 2, 500 μl, vacuum pressure 80% for 180 seconds, once;

Step 3: washing solution 2, 300 μl, vacuum pressure 60% for 120 seconds, two times.

A pre-elution vacuum of 90% pressure is applied for 300 seconds. Hereafter the elution step is performed by the addition of 120 μl elution solution (Applied Biosystems), and the application of a 100% vacuum-pressure for 120 seconds. The RNA samples are collected in 96-well plates (Applied Biosystems). The eluates are split into two aliquots of equal volume. One aliquot is stored at −80° C., the other aliquot is used for RNA amplification and GeneChip analysis.

The RNA biotinylation step involved the use of the High-Yield RNA Labelling Kit (Enzo Diagnostics, NY, USA; P/N 900182) following the manufacturer's instructions. The following ingredients are mixed in an initial step:

22 μl aRNA

4 μl 10×HY reaction buffer,

4 μl 10×Biotin Labeled Ribonucleotides,

4 μl 10×DTT,

4 μl RNase Inhibitor mix,

2 μl 20×T7 RNA polymerase.

The mixture is incubated at 37° C. for 3-4 hours. The labeled aRNA is purified using RNeasy chemistry (Qiagen) following the manufacturer's instructions. The elution volume is 60 μl, 2 μl are used to determine the RNA concentration spectrophotometically by absorbance at 260 nm.

RNA Fragmentation

15 μg labeled aRNA is fragmented in a volume of 20 μl by the addition of 4 μl 5×MES Fragmentation buffer and RNase free water. The mixture is incubated for 20 minutes at 94° C.

12×MES Fragmentation Buffer (for 1000 ml):

| | | |
|---|---|---|
| 70.4 | g | MES free acid (1.22M MES, 0.89M [Na⁺] |
| | | (2-(N-Morpholino)ethanesulfonic acid (SIGMA, P/N M5287) |
| 193.3 | g | MES sodium salt (Sigma, P/N M3885) |
| 800 | ml | DEPC water |

Filter through a 0.2 μm filter, the pH should be between 6.5 and 6.7 without adjustment.

Microarray Hybridization Mix

The hybridization is carried out in a volume of 300 μl. Fragmented aRNA is mixed with 150 μl 2×MES hybridization buffer, 3 μl herring sperm DNA (10mg/ml), 3 μl BSA (50 mg/ml), 3 μl 948b control oligonucleotide (5 nM), and 3 μl 20× Eukaryotic Hybridization Controls (Affymetrix). DEPC water is added to 300 μl final volume.

2×MES Hybridization Buffer (for 500 ml):

| | |
|---|---|
| 217 ml | DEPC water |
| 200 ml | 5M NaCl |
| 82 ml | 12X MES |

Filter through 0.2 μm filter.
Then add: 1.0 ml 10% Triton X-100. Store at room temperature.

Microarray Pre-treatment

The microarray is incubated at 45° C. for 15 minutes. The array chamber is filled with freshly prepared pre-treatment solution, prewarmed to 45° C.

Pre-treatment Solution (300 μl Per Microarrav)

| | |
|---|---|
| 294 μl | 1X MES hybridization buffer |
| 3 μl | Acetylated BSA (50 mg/ml) |
| | (Gibco BRL Life Technologies, P/N 15561-020) |
| 3 μl | Herring sperm DNA (10 mg/ml) |
| | (Promega/Fisher scientific, P/N D1811) |

Microarray Hybridization

RNAs are hybridized to Affymetrix HG U133A chip containing oligonucleotide probes of about 12,000 human genes and analyzed.

While the microarrays are being pre-treated at 45° C., the hybridization mix is incubated at 99° C. for 5 minutes. After a centrifugation for 5 minutes at 14,000 rpm the supernatant is transferred to a new Eppendorf tube and incubated at 45° C. for 5 minutes. The pre-treatment solution is removed from the microarray chamber and replaced with the hybridization mix, avoiding bubbles. The septa of the plastic cartridge are covered with tape and the cartridge is placed in an oven at 45° C. with the glass front facing down. The hybridization is continued for 16 to 18 hours.

Washing Procedure

The hybridization mix is removed from the probe array and set aside in a microcentrifuge tube. 280 μl 1×MES hybridization buffer is added to the chamber and a fluidics wash is performed on a GeneChip Fluidics Station 400 using 6×SSPE-T buffer.

6×SSPE-T wash buffer (1000ml)

300 ml 20X SSPE (BioWhittaker, P/N 16-010Y)

699 ml water

Filter through 0.2 μm filter. Add 1 ml 10% Triton X-100

After the fluidics wash the SSPE-T buffer is removed from the chamber and filled with stringent wash buffer, avoiding bubbles.

Stringent Wash Buffer (1000 ml):

83.3 ml 12×MES buffer

5.2 ml 5M NaCl

1 ml 10% Tween 20

910.5 ml water

Filter through 0.2 μm filter. Add 1 ml 10% Triton X-100.

The microarray cartridges are layed face up in a 45° C. incubation oven for 30 minutes. The stringent buffer is removed and the array is rinsed with 200 μl 1×MES hybridization buffer. The 1×MES hybridization buffer is completely removed, the array chamber filled with SAPE stain, and incubated at 37° C. for 15 minutes.

SAPE Stain (600 μl):

300 μl 2×MES hybridization buffer

288 μl water

6 μl BAS (50 mg/ml)

6 μl SAPE (1 mg/ml) (Molecular probes, P/N 15230-147)

After 15 minutes the SAPE stain solution is removed, the chamber filled with 200 μl 1×MES hybridization buffer, and a fluidics wash is performed. The SSPE-T solution is removed from the microrarray chamber and replaced with 300 μl AB stain.

AB Stain (300 μl):

| | | |
|---|---|---|
| 150 | μl | 2X MES hybridization buffer |
| 146.25 | μl | water |
| 3 | μl | BSA (50 mg/ml) |
| 0.75 | μl | biotinylated antibody (500 μg/ml) |
| | | (Vector laboratories, P/N BA-0500) |

The cartridge is incubated at 37° C. for 30 minutes, the AB stain is replaced with 200 μl 1×MES hybridization buffer, and a fluidics wash is performed. After the wash step, the SSPE-T solution is removed, the chamber is filled with SAPE stain, and incubated at 37° C. for 15 minutes. The SAPE stain is replaced with 200 μl 1×MES hybridization buffer and a fluidics wash is performed. The septa are covered with tape to prevent buffer leakage.

Microarrays are scanned on Affymetrix GeneArray® scanners. Raw data sets are normalized by scaling 75% quantile of all probe sets of each chip to a target intensity of 200.

Data Analysis

Statistical analysis is performed with S-Plus (Insightful, Inc., USA) and GeneSpring 5.0.3^{R} (Silicon Genetics, USA).

In one experiment, genes showing an average expression change superior or equal to 2 and P value <0.001 (parametric test, variances not equal) in the AR or CR groups are selected. This initial filter gives a list of 1434 genes. From this list, the following genes are selected based on their ability to distinguish between control, AR and CR groups, their correlation with the histological signs of acute or chronic rejection, and their ability to be detected in peripheral body fluids.

TABLE 1
List of genes (with GenBank/RefSeq Identifier)
which are indicative of transplant rejection

| GenBank/ | | | | |
|---|---|---|---|---|
| RefSeq | Affymetrix | | fold change | |
| Identifier | probe set | description | AR/C | CR/C |
| BF213829/ | 215051_x_at | allograft inflammatory | 87.2 | 35.5 |
| NM_032955 | | factor-1 | | |
| AU144167/ | 215076_s_at | collagen III alpha 1 | 3.9 | 11.1 |
| NM_000090 | | | | |
| M25915/ | 208791_at | clusterin | 3.8 | 3.0 |
| NM_001831 | | (apolipoprotein J) | | |
| D32039/ | 211571_s_at | versican (chondroitin | 8.3 | 5.8 |
| NM_004385 | | sulfate proteoglycan 2) | | |
| J03040/ | 200665_s_at | osteonectin (secreted | 1.4 | 3.0 |
| NM_003118 | | protein, acidic, | | |
| | | cysteine-rich) | | |
| M83248/ | 209875_s_at | osteopontin (secreted | 7.3 | 3.8 |
| NM_000582 | | phosphoprotein 1, bone | | |
| | | sialoprotein I, early T- | | |
| | | lymphocyte activation 1) | | |
| NM_002423 | 204259_at | Matrix metalloproteinase | 3.2 | 4.4 |
| | | 7 (MMP-7, matrilysln, | | |
| | | uterine) | | |
| NM_004994 | 203936_s_at | Matrix metalloproteinase | 3.5 | 4.4 |
| | | 9 (gelatinase B) | | |
| BC003551/ | 211003_x_at | Transglutaminase 2 (C | 31.8 | 6.9 |
| NM_004613 | | polypeptide, protein- | | |
| | | glutamine-gamma- | | |
| | | glutamyltransferase) | | |

TABLE 2
List of genes (with GenBank accession numbers) indicative of CR

| GenBank/ | | | | | |
|---|---|---|---|---|---|
| RefSeq | Affymetrix | | fold change | | |
| Identifier | probe set | description | AR/C | CR/C | CR/AR |
| M10321/ | 202112_at | von Willebrand factor | −1.5 | 8.3 | 12.5 |
| NM_000552 | | | | | |
| D21254/ | 207173_x_at | OB-cadherin (cadherin 11, | 1.1 | 4.1 | 3.6 |
| NM_033664 | | type 2) | | | |
| D13665/ | 210809_s_at | Osteoblast specific factor 2 | 2.4 | 15.5 | 6.5 |
| NM_006475 | | (fasciclin I-like, OSF-2) | | | |
| U19495/ | 209687_at | Stromal cell-derived factor 1 | −2.0 | 3.1 | 6.6 |
| NM_021704 | | (SDF-1) | | | |
| U88321/ | 210072_at | ELC (exodus-3, small inducible | 2.0 | 15.7 | 7.7 |
| NM_006274 | | cytokine subfamily A (Cys- | | | |
| | | Cys), member 19) | | | |
| M58549/ | 202291_s_at | matrix GLA protein | −1.2 | 4.1 | 4.8 |
| NM_000900 | | | | | |
| U01839.1/ | 208335_s_at | darc (Duffy blood group antigen) | 1.2 | 4.5 | 3.7 |
| NM_002036 | | | | | |
| AF138303/ | 211813_x_at | decorin | 1.2 | 4.3 | 3.5 |
| NM_ 133506 | | | | | |
| K02765/ | 217767_at | C3 complement protein and | 27.6 | 55.7 | 2.1 |
| NM_000064 | | cleavage products | | | |
| M21574/ | 203131_at | PDGF receptor alpha (Platelet- | −1.3 | 3.6 | 4.8 |
| NM_006206 | | derived growth factor receptor, | | | |
| | | alpha polypeptide | | | |

TABLE 3
Gene (with GenBank accession number) indicative of AR

| GenBank/ | | | | |
|---|---|---|---|---|
| RefSeq | Affymetrix | | fold change | |
| Identifier | probe set | description | AR/C | CR/C |
| M34455/ | 210029_at | indolamine-pyrrole 2,3 | 104.2 | 11.1 |
| NM_002164 | | dioxygenase | | |

1. A method of monitoring transplant rejection in a subject comprising
a) taking as a baseline value the level of mRNA expression corresponding to or protein encoded by at least one gene, in a specific tissue sample of a transplanted subject who is known not to develop rejection;
b) detecting a level of mRNA expression corresponding to or protein encoded by the at least one gene identified in a) in an tissue sample of the same tissue type as in a) obtained from a patient post-transplantation; and
c) comparing the first value with the second value, wherein a first value lower or higher than the second value predicts that the transplanted subject is at risk of developing rejection,
wherein the gene is as defined in Table 1, 2 or 3.

2. A method of monitoring transplant rejection in a subject comprising
a) detecting a level of mRNA expression corresponding to or protein encoded by at least one gene, in an tissue sample obtained from the donor at the day of transplantation;
b) detecting a level of mRNA expression corresponding to or protein encoded by the at least one gene identified in a) in an tissue sample obtained from a patient post-transplantation,
c) comparing the first value with the second value, wherein a first value lower or higher than the second value predicts that the transplanted subject is at risk of developing rejection; wherein the gene is as defined in Table 1, 2 or 3.

3. A method for monitoring transplant rejection in a subject at risk thereof comprising
a) obtaining a pre-administration sample from a transplanted subject prior to administration of a rejection inhibiting agent,
b) detecting the level of expression of mRNA corresponding to or protein encoded by the at least one gene in the pre-administration sample,
c) obtaining one or more post-administration samples from the transplanted patient,
d) detecting the level of expression of mRNA corresponding to or protein encoded by the at least one gene in the post-administration sample or samples,
e) comparing the level of expression of mRNA or protein encoded by the at least one gene in the pre-administration sample with the level of expression of mRNA or protein encoded by the at least one gene in the post-administration sample or samples, and
f) adjusting the agent accordingly,
wherein the gene is as defined in Table 1, 2, or 3.

4. A method for preventing, inhibiting, reducing or treating transplant rejection in a subject in need of such treatment comprising administering to the subject a compound that modulates the synthesis, expression or activity of one or more genes or gene products as identified in Table 1, 2, or 3, so that at least one symptom of rejection is ameliorated.

5. A method for identifying agents for use in the prevention, inhibition, reduction or treatment of transplant rejection comprising monitoring the level of mRNA expression of one or more genes or gene products as identified in Table 1, 2, or 3.

6. A method according to claim 1, wherein the transplanted subject is a kidney transplanted subject.

7. A method according to claim 1, wherein the level of expression of the gene expression is assessed by detecting the presence of a protein corresponding to the gene expression product.

8. A method according to claim 7, wherein the presence of the protein is detected using a reagent which specifically binds to the protein.

9. A method according to claim 1, wherein the level of mRNA expression of one or more genes is detected by techniques selected from the group consisting of Northern blot analysis, reverse transcription PCR and real time quantitative PCR.

10. A method according to claim 1 wherein the level of mRNA expression of a set of genes is detected.

11-12. (canceled)

13. A method or use according to claim 1, wherein the transplant rejection is chronic transplant rejection and the gene is as defined in Table 2.

14. A method or use according to claim 1, wherein the transplant rejection is acute transplant rejection and the gene is indolamine deoxygenase.

15. A method according to claims 1, wherein the tissue sample is a body fluid.
